(19) United States
(12) Patent Application Publication
(10) Pub. No.: US 2015/0272774 A1
(43) Pub. Date: Oct. 1, 2015

(54) MASSAGE TOOL

(71) Applicant: Wen-Hwa LEE, Hsinchu County (TW)

(72) Inventor: Wen-Hwa LEE, Hsinchu County (TW)

(21) Appl. No.: 14/477,181

(22) Filed: Sep. 4, 2014

Related U.S. Application Data
(60) Provisional application No. 61/970,148, filed on Mar. 25, 2014.

(51) Int. Cl. A61F 7/00; A61H 99/00

(52) U.S. Cl. CPC A61F 7/00; A61H 99/00; A61H 2201/0207

(57) ABSTRACT

A massage tool includes a main body; and an active member coupled to the main body. The active member includes an active material capable of being heated for transferring heat to the main body so as to activate or reactivate the massage tool. If necessary, more than one active material may be included in the active member. Heat can be transferred from the active material to the main body by way of conduction of the active material or convection of an active vapor released from the active material. The active vapor, for example, can be steam for providing heat and/or vaporized essential oil for aromatic treatment. The active material, for example, can be heated by an external heating source or an internal heating source.

### CROSS-REFERENCE TO RELATED APPLICATIONS

The present application is a non-provisional application claiming benefit from a prior-filed provisional application bearing a Ser. No. 61/970,148 and filed Mar. 25, 2014, the entity of which is incorporated herein for reference.

### FIELD OF THE INVENTION

The present invention relates to a massage tool, and particularly to a massage tool which is capable of being activated and reactivated.

### BACKGROUND OF THE INVENTION

Trigger points, also known as trigger sites or muscle knots, are hyperirritable spots in skeletal muscle of a human body, which are associated with palpable nodules in taut bands of muscle fibers. Generally, myofascial pain can be relieved to some extent by exerting pressure or treatment to these points. Therefore, a variety of massage balls have been developed for this purpose and are used for stimulating one or more trigger points. A conventional massage ball has a spherical main body with a coarse surface for stimulating trigger points of a human body. The conventional massage ball functions simply by traveling on the human body and pressing the triggering points. The effect and efficiency of the treatment are not satisfactory.

### SUMMARY OF THE INVENTION

The present invention relates to a massage tool, which functions in a more efficient way and achieves better performance than prior art.

The present invention relates to a massage tool, which can be activated and reactivated so as to be applied in a variety of fields with low cost.

The present invention relates to a massage tool, which includes a main body; and an active member coupled to the main body and including an active material capable of being heated for transferring heat to the main body so as to activate or reactivate the massage tool.

In an embodiment, the active member is contiguous with the main body so that heat is transferred from the active material to the main body by way of heat or thermal conduction. The term “heat or thermal conduction” used herein substantially means heat transfer caused by microscopic diffusion and collisions of particles or quasi-particles within the massage tool.

In another embodiment, heat is transferred from the active material to the main body by way of convection of an active vapor released from the active material.

If necessary, more than one active material may be included in the active member. The active vapor, for example, can be steam for providing heat and/or vaporized essential oil for aromatic treatment.

The active material, for example, can be heated by an external heating source or an internal heating source.

### BRIEF DESCRIPTION OF THE DRAWINGS

The above objects and advantages of the present invention will become more readily apparent to those ordinarily skilled in the art after reviewing the following detailed description and accompanying drawings, in which:

FIG. 1 is a schematic diagram illustrating a massage tool according to a first embodiment of the present invention;

FIG. 2 is a schematic diagram illustrating a massage tool according to a second embodiment of the present invention;

FIG. 3 is a schematic diagram illustrating a massage tool according to a third embodiment of the present invention; and

FIG. 4 is a schematic diagram illustrating a massage tool according to a fourth embodiment of the present invention;

FIG. 5A and FIG. 5B are schematic cross-sectional views illustrating examples of a massage tool according to a fifth embodiment of the present invention;

FIG. 6A is a schematic block diagram of a circuit module included in a massage tool according to a sixth embodiment of the present invention; and

FIG. 6B is a schematic diagram illustrating the appearance of the sixth embodiment of massage tool according to the present invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for the purpose of illustration and description only. It is not intended to be exhaustive or to be limited to the precise form disclosed.

A massage tool, if applicable to acupuncture and moxibustion therapy, would be more helpful than just for pressing trigger points. Therefore, the present invention provides a massage tool capable of being applied to acupuncture and moxibustion therapy for trigger point treatment as well as general massages for relaxation or special treatment such as aromatherapy or herbal treatment. For achieving the objectives, a massage tool according to the present invention includes a main body and one or more active members coupled to the main body. The active members are capable of generating heat so as to raise the temperature of the massage tool and/or release vapor of essential oil or other volatile ingredients so as to activate or reactivate the massage tool and thus facilitate treatment effect, e.g. a variety of aromatic treatments. The main body can be of any suitable shape and configurations adapted for receiving and supporting the active member.

Hereinafter, embodiments and examples of massage tools according to the present invention are given for the above-mentioned features.

Please refer to FIG. 1. A massage tool 1 according to a first embodiment of the present invention includes a container 11 as the main body and at least one core 12 as the active member. The container 11 is of a spherical shape and composed of two hollow semi-spherical portions 111 and 112. The semi-spherical portions 111 and 112 have openings 1110 and 1120 in the junction faces and are engageable with and disengageable from each other with coupling parts (not shown). The two hollow semi-spherical portions 111 and 112 can be assembled in a variety of ways. The coupling parts, for example, can be outer threaded structure and inner threaded structure matching with each other. Preferably, the coupling parts are integrally formed with the two hollow semi-spherical portions 111 and 112, respectively, and particularly designed to have the two hollow semi-spherical portions 111 and 112 identical to each other. This is advantageous as a single mode can be used for producing both the portions 111 and 112. By separating the semi-spherical portions 111 and 112, the core 12 can be filled into the container 11 from the openings 1110 and 1120, and if necessary, the core may be replaced or changed with other core material. The massage tool 1 is capable of being activated to generate heat and/or release active vapor by heating the core 12, for example, through microwave oven heating. To be heated with a microwave oven, the material of the core may contain moisture, e.g. grains, and steam is generated for providing heat for the massage tool 1. To release active vapor, the material of the core is one or more volatile ingredients, e.g. essential oil(s), or may absorb or adhere volatile ingredients. The core material can be filled into the container directly or wrapped with a gas permeable bag or cloth in advance. Different kinds of cores may co-exist in the room of the container 11 and heated with a microwave oven or similar or alternative heating sources at the same time. Accordingly, the temperature of the main body increases with the heated core(s), and the massage tool is thus activated. Once the massage tool is cooling down with passing time, the core(s) can be replaced and heated again to heat the main body so that the massage tool can be reactivated.

In addition to the above-described, the massage tool may optionally exhibits one or more other features for further improvement. Described below are some of the examples.

For releasing heat and/or vapor, it is desirable that a plurality of orifices are provided on the container 11 for dissipation of heat and/or vapor.

To avoid explosion due to high vapor pressure inside the container 11, it is desirable that one or more air vent valves are provided on the container 11 for modulating gas pressure inside the container 11.

To prevent from overheating to a dangerous high temperature, it is desirable that a temperature indicator is provided. In an example, the temperature indicator 115 is mounted onto the container 11 in a form of a small piece. In a specific example, the temperature indicator 115 is an area of the container 11 coated with a temperature ink whose color changes with temperature. Alternatively, the container 11 itself is made of, mixed with, or wrapped with a material whose color varies with temperature or exhibits any other temperature-dependent feature. For general purpose, the massage ball in this embodiment may reach an inner core temperature around 130 degrees C. and surface temperature up to 60 degrees C. after being heated with a microwave oven under a power of 1100W for 30 seconds. The heated massage ball may be kept at 40-60 degrees C. for at least 30 minutes. In response to practical requirements and depending on materials of active member and main body, the temperature range may vary by changing the heating power and time.

For safety, it is desirable that the container 11 is made of a specific material and having a specific structure strong enough for bearing the force exerted thereon, e.g. the weight of a physician. Moreover, it is desirable that the container 11 has a proper heat-conducting capability so as to improve treatment efficiency without burning the patient. For example, the material of the container 11 includes, but not limited to, injection-molded polypropylene (PP), densely weaved cotton or hemp, or silicon dioxide (silicon gel).

For simulating massage with fingers, the outer surface of the container 11 is made irregular. For example, irregular protruding members (not shown) may be mounted onto the surface of the container 11 or integrally formed with the container 11.

In this embodiment, the heating of the core 12 as well as the container 11 is made by way of passive heating such as microwave oven heating. Therefore, the heating of the core 12 can be repetitively performed and thus the massage tool 1 can be reactivated. An example of the core 12 in the passive heating case is grains Examples of grain include, but not limited to, red beans, mung beans, soybeans, rice wheat, buckwheat or flax seed. Alternatively, active heating may also be used for implementing the invention. For example, chemical material which may actively generate heat, for example by shaking, squeezing or blending, is used for heating the core 12. The active heating material, for example, may also be a disposable or one-time-use warmer.

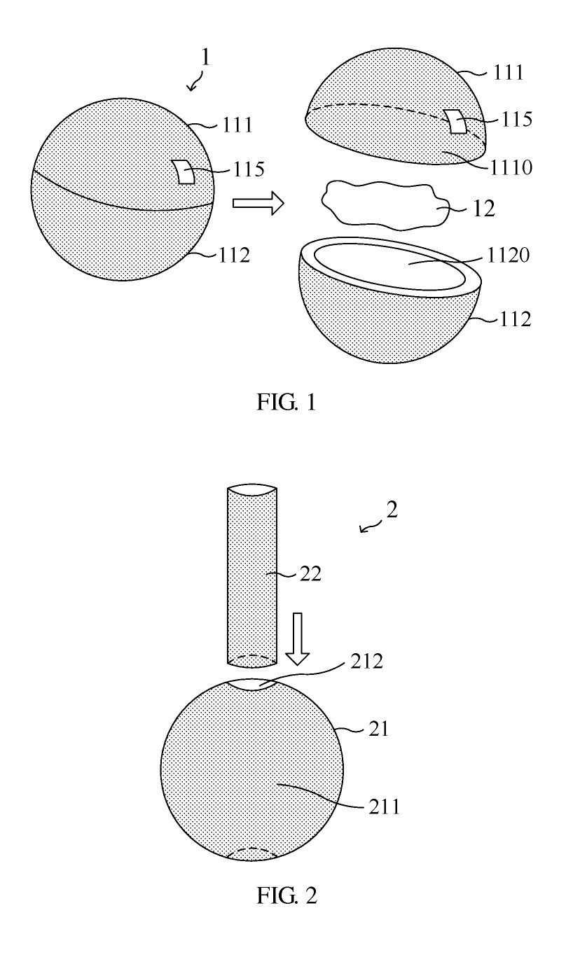

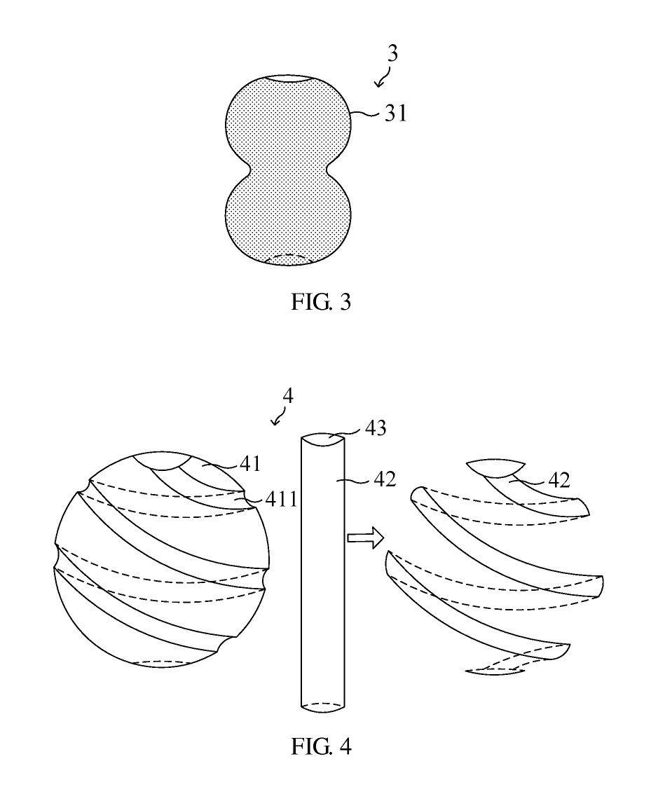

A second embodiment of massage tool 2 according to the present invention, as shown in FIG. 2, is similar to the first embodiment of massage tool 1 with some modifications. The container 21 in this embodiment is a hollow ball integrally formed, e.g. by way of injection molding, and having a room 211 thereinside for accommodating the core 22. The room 211 may have one opening or two openings at both ends thereof. The core 22 can be a relatively hard gas permeable bag containing core material, and having such a specific dimension as to well fit the room 211 when being inserted into the room 211. In another example, the core 22 can be a soft gas permeable bag or cloth containing core material, and placed into the room 211 via an opening 212 formed on the hollow ball 21 and then sealed with a cover (not shown). The size of the opening 212 is relevant to the size of the core 12.

In a third embodiment of massage tool 3 according to the present invention, as shown in FIG. 3, the container 31 is of a peanut shape instead of a spherical shape. The container may be composed of two or more parts like the first embodiment or integrally formed like the second embodiment. The configurations, material and optional elements included in the massage tool 3 may be the same as or similar to those described in the first embodiment and shown in FIG. 1. Of course, depending on practical designs, the container 31 can be of other shapes adapted for different parts of the body, e.g. neck, shoulders, back, chest, arms, waist, legs, etc. For example, the massage tool may alternatively a massage roller, a massage cake, a massage peanuts, a massage ring, a massage dumbbell, etc. The peanut-shaped massage tool as shown in FIG. 3 is particularly advantageous in treating a user's neck.

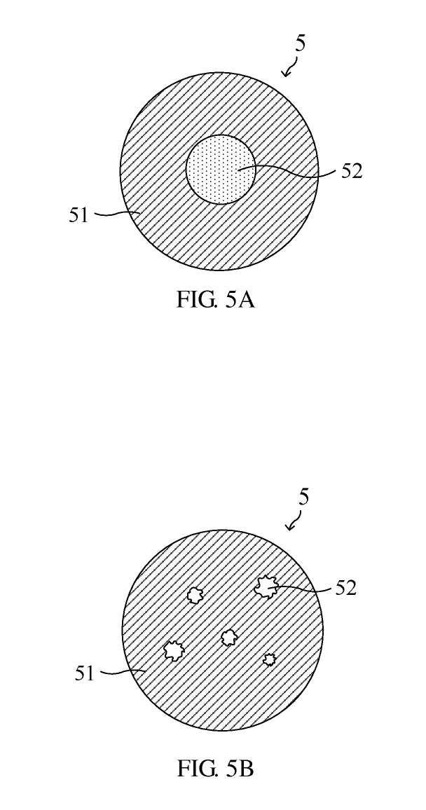

A fourth embodiment of massage tool 4 according to the present invention, as shown in FIG. 4, is similar to the first, second or third embodiment of massage tool with some modifications. In this embodiment, the active member 42 is attached onto the surface of the main body 41, e.g. the outer surface, instead of being filled into the inner space of the main body 41. Therefore, the main body 41 in this embodiment may be an integrally formed solid body. On the outer surface, depressions 411 are formed, for example continuously configured as a helix. The active member 42 can be an elongated soft gas permeable bag or cloth containing active material, and embedded into the depressions. A temperature indicator 43 as described above can be arranged, for example, at one or both surfaces or any other suitable surfaces of the active member and embedded onto the outer surface of the main body 41 to be observed.

The present invention further provides a fifth embodiment of massage tool 5, which includes a main body 51 and an active member 52, as shown in FIG. 5A and 5B. The active member 52 includes an inorganic material as the active material. For example, salt or a ceramic material can be used as the active material, and wrapped with or inserted in the main body 51 so as to be contiguous with the main body 51. The ceramic material can be, for example but not limited to, silicon carbide (SiC). FIG. 5A is a schematic cross-sectional view of an example of the massage tool 5, in which the active member includes a mass of inorganic material. FIG. 5B is a schematic cross-sectional view of another example of the massage tool 5, in which the active member includes dispersed inorganic material. In either way, when the active material is heated, heat can be transferred from the active material to the main body by way of heat or thermal conduction. The term “heat or thermal conduction” used herein substantially means heat transfer caused by microscopic diffusion and collisions of particles or quasi-particles within the massage tool. Accordingly, the temperature of the main body is raised, and the massage tool is thus activated. Once the massage tool is cooling down with passing time, the active material can be heated again to heat the main body so that the massage tool can be reactivated. Depending on practical designs, the main body 51 can be integrally formed of any suitable shapes adapted for different parts of the body, e.g. neck, shoulders, back, chest, arms, waist, legs, etc. For example, the massage tool may be a massage ball, a massage roller, a massage cake, a massage peanuts, a massage ring, a massage dumbbell, etc. The peanut-shaped massage tool as shown in FIG. 3 is particularly advantageous in treating a user's neck.

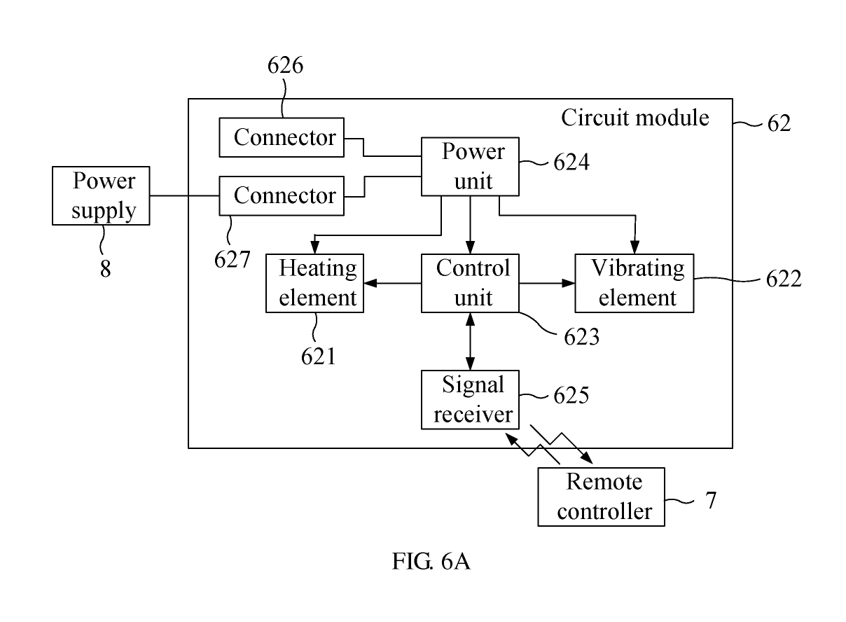

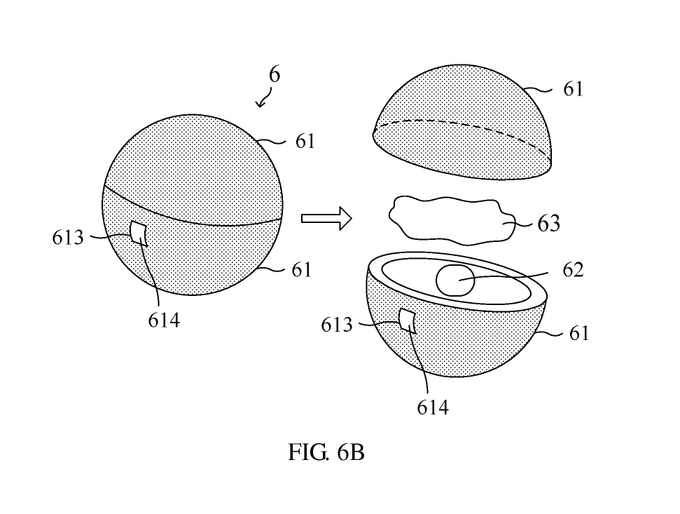

In the above embodiments, the massage tools are activated or reactivated by heating the core material with an external heating source such as a microwave oven or an internal heating source such as specific chemical material. In a sixth embodiment of the massage tool according to the present invention, the structure and material of each part of the massage tool can be similar to any of the equivalent parts exemplified in the above embodiments. The sixth embodiment of massage tool differs from the above embodiments in comprising an internal heating source to activate or reactivate the massage tool. Moreover, it is known that simultaneously heating and vibrating a massage tool may significantly improve the effect of certain treatment. Therefore, a vibration function is preferably introduced into the massage tool. For achieving these objects, some elements are introduced. For illustration purpose, the massage tool as described in the first embodiment is referred to. It is to be noted that the introduced elements in this embodiment may be used with any other suitable embodiments according to the present invention. In this embodiment, the massage tool 6 further includes a circuit module 62 accommodated in a container 61 thereof. The circuit module 62 includes a heating element 621, a vibrating element 622, a control circuit 623 in communication with the heating element 621 and the vibrating element 622, and a power unit 624 electrically connected to the heating element 621, as shown in FIG. 6A. In this embodiment, the massage tool 6 is activated or reactivated to generate heat with the heating element 621. The heating element 621 can be any proper electric heater capable of converting electrical energy to heat. For example, it can be but is not to be limited to an electrical resistor. The control unit 623 optionally actuates the heating element 621 while the massage tool 6 is in use and controls the heating temperature within an effective temperature range to avoid burning the user. On the other hand, the massage tool 6 can also be activated or reactivated to generate vibration with the vibrating element 622. The vibrating element 622, for example, can be but is not to be limited to an Eccentric Rotating Mass vibration motor. The control unit 623 also optionally actuates the vibrating element 622 with or without heating. The control unit 623, for example, can be but is not to be limited to a microcontroller unit. The power unit 624 supplies power for the work of the heating element 621, the vibrating element 622 and the control circuit 623. The power unit 624, for example, can be but is not to be limited to a battery or a rechargeable battery.

In this embodiment, the heating element 621 and the vibrating element 622 are both included in the massage tool 6. Alternatively, treatment can be performed with heating or vibrating only. For this purpose, the heating element 621 and the vibrating element 622 may exist or work independently.

Furthermore, the massage tool 6 may be remotely controlled with a remote controller 7. The remote controller 7, for example, can be a radio frequency (RF) remote controller or an infrared ray (IR) remote controller. Alternatively, the remote controller 7 can also be implemented with any other existent wireless communication device, e.g. a smart phone, a PC tablet or a PC. For communicating with the remote controller 7, the circuit module 62 further includes a signal receiver 625 for receiving the control signal transmitted by the remote controller 7. The type of the signal receiver 625 depends on the type of the remote controller 7. For example, if the remote controller 7 is an RF or IR remote controller, the signal receiver 625 will be an RF or IR receiver. On the other hand, by installing an application program specific to the massage tool 6, the existent wireless communication device 7 may build up wireless communication with the signal receiver 625, for example, by way of Bluetooth® short range wireless technology or Wi-Fi® wireless local area network module thereof.

In an extensive embodiment, the massage tool 6 may also include a core similar to any of the cores exemplified above. FIG. 5B schematically illustrates an example of the core 63 similar to the core 12 shown in FIG. 1. Furthermore, for illustration only, an example of the container 61 is the container 11 shown in FIG. 1 and its variation. However, any of other suitable embodiments of the main body can be used in replace of the container 61. In this embodiment, the container 61 may have an opening 613 covered with a removable lid 614. The opening 613 exposes a connector 626 coupled to the control unit 623. Via the connector 626, an external equipment such as the remote controller 7 or any other suitable device may communicate with the circuit module 62 of the massage tool 6 for specified functions. For example, the connector 626 is electrically connected to the power unit 624, so the external device may acquire power from the power unit 624. In other words, the massage tool 6 may additionally serve as a battery bank if it is not in use. The connector 626, for example, can be but is not to be limited to a USB connector.

Furthermore, if a rechargeable battery is used as the power unit 624, the circuit module 62 may further includes a connector 627 for connecting the power unit 624 to an external power supply 8 for recharging. The connector 627 may also be exposed from the opening 613

By way of the above embodiments and examples and from the above descriptions, it is understood that a massage tool according to the present invention can be activated and reactivated by heating and/or vibrating in an easy and cost-efficient way. With the circuit module, the massage tool may serve as a battery bank when it is not in use so as to make use of the device. Furthermore, the remote control of the massage tool make it convenient for use.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the invention which are to be accorded with the broadest interpretation so as to encompass all such modifications and similar structures.

What is claimed is:

1. A massage tool, comprising:
a main body; and
an active member coupled to the main body and including an active material capable of being heated for transferring heat to the main body so as to activate or reactivate the massage tool.

2. The massage tool according to claim 1 wherein the active member is contiguous with the main body so that heat is transferred from the active material to the main body by way of conduction.

3. The massage tool according to claim 2 wherein the active material is an inorganic material.

4. The massage tool according to claim 3 wherein the active material is salt or ceramic.

5. The massage tool according to claim 1 wherein heat is transferred from the active material to the main body by way of convection of an active vapor released from the active material.

6. The massage tool according to claim 5 wherein the main body has a plurality of orifices for dissipation the active vapor.

7. The massage tool according to claim 5 wherein the active member further includes a gas permeable bag or cloth for wrapping the active material and allowing the active vapor to escape therefrom.

8. The massage tool according to claim 5 wherein the active material includes a moisture-containing material, and the active vapor includes steam for providing heat.

9. The massage tool according to claim 1 wherein the main body is integrally formed with the active member inserted therein.

10. The massage tool according to claim 1 wherein the main body includes a container composed of a first semi-spherical hollow portion and a second semi-spherical hollow portion engageable with each other for accommodating the active material therein and disengageable from each other for replacing the active material.

11. The massage tool according to claim 1 wherein the main body includes a container having a room and an opening for filling and replacing therefrom the active material.

12. The massage tool according to claim 1 wherein the main body includes an elongated depression formed on an outer surface of the main body and configured as a helix, and the active material is embedded into the depression.

13. The massage tool according to claim 1, further comprising a temperature indicator for indicating temperature changes.

14. The massage tool according to claim 13 wherein the main body is made of or mixed with a material whose color changes with temperature; the main body is coated with a thermal-sensitive ink whose color changes with temperature; or the main body is wrapped with a material whose color changes with temperature, so as to serve as the temperature indicator.

15. The massage tool according to claim 1 wherein the active member includes more than one kind of active material.

16. The massage tool according to claim 1 wherein the active material is capable of being heated by an external heating source.

17. The massage tool according to claim 16 wherein the external heating source is microwave oven.

18. The massage tool according to claim 1, further comprising an internal heating source accommodated in the main body for heating the active material.

19. The massage tool according to claim 18 wherein the internal heating source is a disposable and replaceable warmer containing chemical material which actively generates heat by shaking, squeezing or blending.

20. The massage tool according to claim 18 wherein the internal heating source is a circuit module including:
a heating element for heating the active material;
a control circuit in communication with the heating element for controlling operations of the heating element; and
a power unit electrically connected to the heating element and the control unit for providing power for the operations of the heating element.

21. The massage tool according to claim 20 wherein the circuit module further includes a vibrating element in communication with the control unit and electrically connected to the power unit for vibrating the main body under the control of the control unit.

22. The massage tool according to claim 20 wherein the circuit module further includes a signal receiver in communication with the control unit for receiving a control signal transmitted by a remote controller to remotely control the heating of the active material.

23. The massage tool according to claim 22 wherein the remote controller is a radio frequency (RF) or infrared ray (IR) remote controller, the signal receiver is an RF or IR signal receiver.

24. The massage tool according to claim 22 wherein the remote controller is a wireless communication device installed therein an application program specific to the massage tool and building up wireless communication with the signal receiver by way of Bluetooth® short range wireless technology or Wi-Fi® wireless local area network transmission.

25. The massage tool according to claim 20 wherein the circuit module further includes a first connector electrically connected to the power unit and communicable with an external device for transmitting power from the power unit to the external device.

26. The massage tool according to claim 25 wherein the main body has an opening covered with a removable lid, and the first connector is exposed for connecting to the external device when the opening is uncovered from the removable lid.

27. The massage tool according to claim 20 wherein the circuit module further includes a second connector electrically connected to the power unit and communicable with an external power supply for recharging the power unit.
